(19) United States
(12) Patent Application Publication
(10) Pub. No.: US 2006/0184240 A1
(43) Pub. Date: Aug. 17, 2006

(54) Annuloplasty chain

(75) Inventors: Jorge Hernan Jimenez, Atlanta, GA (US); Ajit P. Yoganathan, Tucker, GA (US); Zhaoming He, Lubbock, TX (US)

Correspondence Address: TROUTMAN SANDERS LLP, 600 PEACHTREE STREET , NE, ATLANTA, GA, 30308, US

(73) Assignee: Georgia Tech Research Corporation, Atlanta, GA (US)

(21) Appl. No.: 10/561,900

(22) PCT Filed: Jun. 25, 2004
(86) PCT No.: PCT/US04/20219

§ 371 Date: Dec. 22, 2005

Related U.S. Application Data
(60) Provisional application No. 60/482,393, filed on Jun. 25, 2003.

(51) Int. Cl. A61F 2/24

(52) U.S. Cl. 623/2.36

(57) ABSTRACT

An annuloplasty chain (10) is disclosed. The chain (10) can be fabricated from metal, and include a shielding layer (60) and a suturing layer (80).

### BENEFIT CLAIMS TO PRIOR APPLICATIONS

This application claims the benefit of U.S. Provisional Application No. 60/482,393, filed 25 Jun. 2003.

### GOVERMENT INTERESTS

This invention was made in part during work supported by the U.S. Government, including grants from the National Institutes of Health (NIH) E17-649 and HL52009. The government may have certain rights in the invention.

### BACKGROUND OF THE INVENTION

1. Field of the Invention

This invention relates generally to the field of prostheses for cardiac valve repair, and specifically to an annuloplasty implant device incorporating a chain.

2. Description of Related Art

A frequently used method for eliminating some pathological alterations of the mitral and tricuspid valves of the heart is that of reinstating the correct shape and dimensions of the valve annulus by means of surgical procedures known as annuloplasty. Annuloplasty includes surgically implanting a supporting prosthesis on the dilated or deformed annulus for the purpose of reinstating its dimensions and/or physiological shape in such a way as to allow the cardiac valve to function correctly.

Support prostheses utilized in valve repair operations are sometimes called annuloplasty rings. An annuloplasty ring can be implanted around the mitral or tricuspid heart valve for reconstructive treatment of valvular insufficiency. Annular dilation or degradation may influence valve function causing cardiac insufficiency under specific pathologies.

Regeneration of the shape of the mitral annulus has been shown to be beneficial in restoring valve function. There are currently more than 25 different annuloplasty devices on the market. The main types of rings include rigid, flexible, “partial” flexible, and adjustable.

Rigid type rings are widely employed with success, and reduce the dilatation of the valve annulus. Such rings generally include a metal core (for example, a titanium alloy), an optional sheath of cladding around the core, and an outer cladding of textile for suturing. Rigid rings generally do not allow the annulus of the valve to flex along the base of the posterior cuspid in such a way as to assist the cardiac muscle movements. Consequently, significant stress is imposed on the suture points subjected to torsion and traction, which prevents natural behavior of the valve.

Unlike rigid rings, flexible rings follow the movements of the annulus during the cardiac cycle in a beneficial manner. Flexible rings interfere less with normal mitral valve motion, improve peak velocity across the ring, and thus improve ventricular end-diastolic diameter and volume. However, they too have the disadvantage of not allowing the shape to be reconstructed in an optimal manner.

“Partial” flexible rings seek to unite the advantages of the rigid type with those of the completely flexible type while avoiding the disadvantages of each. Theoretically, they are easier and quicker to insert since no sutures have to be placed in the anterior annulus.

Adjustable rings are designed to allow for adjustment of the annular length during valve testing.

Thus, while many conventional rings may restore annulus shape, annular dynamics are lost when using rigid rings, and there remains controversy on the efficiency of flexible rings in preserving these dynamics.

Annular flexing and contraction is likely of importance in valve efficiency, not only mechanically, but functionally. Therefore, an annuloplasty device that minimally interferes with annular dynamics would be an improvement over current annuloplasty ring technology. It can be seen that there is a need in the art for an improved annuloplasty chain that maximally preserves annular dynamics in use.

Further, conventional rings are known to be hard to bend to distort their shape so they can be delivered. It would be beneficial to provide a device that is advantageous in minimally invasive procedures. Thus, it can also be seen that there is a need in the art for an improved annuloplasty chain that is easier to arrange than the conventional ring, so it fits in a minimally invasive delivery system.

Additionally, conventional rings are known to be difficult to use in beating heart procedures. It would be beneficial to provide a device that is advantageous in minimally invasive procedures that can thus be used in beating heart surgeries. It can therefore also be seen that there is a need in the art for an improved annuloplasty chain that can be used in a minimally invasive delivery system so that be implanted in procedures with a beating heart. Beating heart surgeries improve patient survival and reduce surgical complications.

In essence, the present annuloplasty implant device opens new fields of implants for annuloplasty repair since conventional implants are rings.

### BRIEF SUMMARY OF THE INVENTION

The present invention comprises an annuloplasty chain of metal, the chain having a surrounding shielding layer and a suturing layer. A re-sterilizable chain holder can be used during implant of the annuloplasty chain.

The present chain is a solution to the disadvantages inherent in conventional rings. The chain reconstructs the shape of the annulus, while maintaining the dynamics of the valve through appropriate flex and bend. The annuloplasty chain preserves a three-dimensional perimeter, enabling it to adjust the size of the annulus to a fixed quantity after dilation or degradation.

The present chain can be implanted in minimally invasive procedures, and thus in beating heart procedures. The present chain can preferably achieve the complete saddle shape of the annulus with a 1/3 height-to-commissural diameter ratio, and has the ability to maintain a normal chordal force distribution as its bending is dominated by its mechanical environment.

In preferred embodiments, the present annuloplasty chain comprises a multilink chain, a solid link chain or a scaled chain. The chain is preferably fabricated from metal having favorable characteristics of wear under cyclic loading and friction, biocompatibility, tensile strength, and MRI safety. The links of the chain can include links of varying sizes and shapes for improved function with specific pathologies, or may include links of uniform sizes and shapes.

The chain is covered with a flexible, biocompatible polymer layer, which will isolate blood from the device. This shielding layer prevents blood damage, and therefore thrombogenesis.

The shielding layer can be covered by a suturing layer of preferably cloth to enable suturing of the ring.

The chain holder dictates the initial shape of the chain, and the size of the implant. The surgeon should be able to suture the chain completely around the valve before retrieving the holder.

In vitro testing has been conducted to observe the mechanical and functional implications of a saddle-shaped annulus. Testing is also being conducted to elucidate the importance of annular dynamics on chordae tendinea mechanics. Initial results using embodiments of the present invention have shown that valve function is preserved for the range of annular geometries generated by the multilink chain saddle-shaped annulus. This implies that the extreme geometries generated by a multilink chain allow the valve to seal with no significant mitral regurgitation observed.

The annuloplasty implant of the present invention allows the surgeons to have a truly flexible instrument that will preserve the natural dynamic characteristics of the mitral annulus, as they have shown to be important in valve function.

Further, annuloplasty implant of the present invention can preferably achieve the complete saddle shape of the annulus with a 1/3 height-to-commissural diameter ratio.

The present annuloplasty chain can further be utilized as a delivery system. The chain can have a link, or a plurality of links, having an internal cavity or cavities, or being formed of a material with porosity or a material composition that will enable the link(s) to store a pharmaceutical agent or other substance necessary for patient treatment. Substances can include solids, liquids or gases that can be release from within the link(s) in a controlled fashion after the implantation of the device. The substance can include monitoring elements like electronics to send environmental characteristics about the chain or surrounding areas to a doctor, and thus the substance is not intended to exit the link, or a medicinal substance that is designed to exit the surface of a link, or from within a link. Alternatively, the substance can be a refrigerant or the like that simply keeps at least portions of the implant cool. Thus, one delivery system embodiment of the annuloplasty chain can be a drug delivery system in addition to its normal function as a cardiac prosthesis. Other delivery systems can include the ability to provide temperature control to surrounding areas, or the chain can have monitoring means to deliver monitoring characteristics to a doctor.

These and other objects, features and advantages of the present invention will become more apparent upon reading the following specification in conjunction with the accompanying drawing figures.

### BRIEF DESCRIPTION OF THE FIGURES

FIG. 1 illustrates the present annuloplasty implant device comprising a multilink chain according to a preferred embodiment of the present invention.

FIG. 2 illustrates the present annuloplasty implant device comprising a solid link chain according to a preferred embodiment of the present invention.

FIG. 3 illustrates the present annuloplasty implant device comprising a scaled chain according to a preferred embodiment of the present invention.

FIG. 4 illustrates the shielding layer and suturing layer of present annuloplasty implant device according to a preferred embodiment of the present invention.

FIG. 5 illustrates an attachment system with attachment devices of present annuloplasty implant device according to a preferred embodiment of the present invention.

FIG. 6(a) shows a mitral valve sutured on a flexible membrane.

FIG. 6(b) shows the saddle configuration is present when the basal chords are extended as observed by a tracing over the annulus.

FIG. 7 is a schematic of the Georgia Tech left heart simulator.

FIG. 8 is a schematic of the saddle shape configuration setup and local orientation.

FIG. 9 is a diagram of an extended mitral valve identifying the chordae tendineae selected for tension measurements.

FIG. 10(a) is a diagram of the chordae tendineae insertion pattern.

FIG. 10(b) is a lateral diagram of the mitral valve with average chordal lengths.

FIG. 11 are pressure and chordae tendineae tension curves for valve #6 in flat annulus configuration.

FIG. 12 are pressure and chordae tendineae tension curves for valve #6 in saddled annulus configuration.

FIG. 13 are pressure vectors acting on the mitral valve anterior leaflet in a flat and saddled configuration. Pressure vectors are redirected towards the sides of the valve in the saddle configuration decreasing the resultant force in the direction of the anterior strut.

### DETAILED DESCRIPTION OF PREFERRED EMBODIMENTS

Referring now in detail to the drawing figures, wherein like reference numerals represent like parts throughout the several views, the present invention is a medical device comprises an annuloplasty chain 10 of metal, a shielding layer 60, a suturing layer 80, and an attachment system 90 to facilitate attachment of the chain 10 to annulus tissue. The chain 10 is capable of generating a three-dimensional saddle shape while maintaining its perimeter relatively constant. Thus, it maintains annular dynamics while correcting annular size after valvular dilatation.

The chain 10 maintains a relatively constant three-dimensional constant, preferably approximately 3% maximum deformation; thus, the present invention can correct annular degradation. The chain 10 is able to generate saddle-shaped annulus geometries with a saddle height to commissural ratio of up to approximately 25%.

The chain 10 is preferably fabricated from metal, but can be fabricated from other materials, or combinations of materials, that have favorable characteristics of wear under cyclic loading and friction, biocompatibility, tensile strength, and MRI safety.

In preferred embodiments, the present annuloplasty chain 10 comprises a multilink chain 12, a solid link chain 22 or a scaled chain 42. These specific designs preserve a three-dimensional perimeter.

Adjacent links of the chain can be movable relative to one another, have a fixed orientation to each other, or a single chain can have links both movable and fixed. The links can be fabricated so movement between adjacent links are controlled without additional means to aid in such movement, or the joints between links can incorporate additional means to control such movement, other than the contact point(s) between links. For example, the solid link chain 22 embodiment can utilize pins between adjacent links.

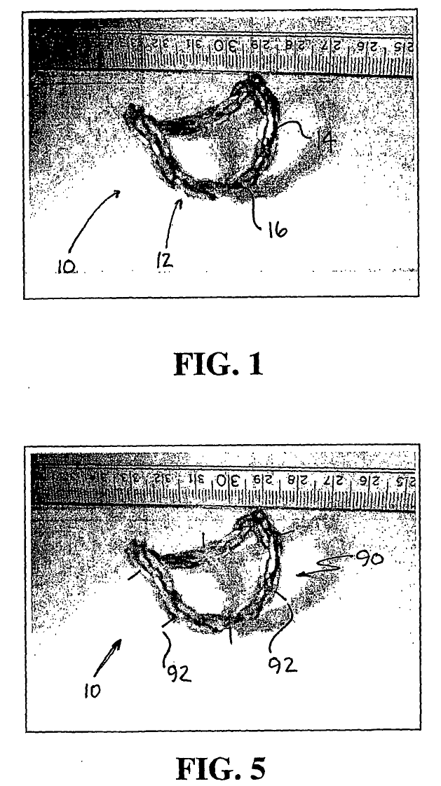

As shown in FIG. 1, a multilink chain 12 incorporates several links 14, wherein the multilink chain 12 is able to generate a saddle-shaped geometry while maintaining its three-dimensional perimeter significantly constant, wherein the perimeter variation is approximately 3%. This chain embodiment is of generally simple construction, but may use a large number of joints 16, which can be welded joints. Yet, welded joints can lead to a greater possibility of failure if not welded appropriately.

A solid link chain 22 is shown in FIG. 2. Chain 22 comprises solid links 24 joined at a pivot 26. The pivot 26 can incorporate cooperating members 28 from adjacent links 24 with a pin 32 rotationally connecting the members 22 to one another. The term “solid link” in this embodiment does not infer that the link 24 is solid throughout, but that it has a distinguishing design from that of an ordinary chain link 14 designed as a loop, as shown in FIG. 1. Solid link 24 may be solid throughout its cross-section, although the links 24 may have cavities therein. Such internal cavities can be filled, partially or totally, with elastomeric material, in particular silicone, polyurethane and their mixtures.

The pivot direction can rotate from one link to another to allow three-dimensional deformations in order to produce the saddle configuration. This chain 22 has a generally approximately negligible variation in perimeter, which is defined by the fit between the different members 28. This design preferably has no welded joints, but the pins 32 used with the members 28 are susceptible to wear because of the high frequency of the loading on the chain 22.

In yet another embodiment, the present annuloplasty chain 10 comprises scaled chain 42 as shown in FIG. 3. This design resembles that used in a key chain, characterized by a relatively smooth surface 44 with the hinge points (not shown) within the surface 44. Because this design has a smooth surface 44, and its hinge points are not exposed, it causes less blood damage due to moving parts. The perimeter change for this design is on the order of approximately 2%.

The chain 10 is preferably a self-lubricating metallic fabricated from surgical steel or titanium, having favorable characteristics of wear under cyclic loading and friction, biocompatibility, tensile strength, and MRI safety. The chain 10 can alternatively be made from materials such as Elgiloy (a cobalt-nickel alloy), titanium, or Nitinol (a nickel-titanium alloy).

The present chain can further be utilized as a delivery system of monitoring characteristics, or drugs, or cooling, among other delivery embodiments. The chain can have a link, or a plurality of links, having a coating of a substance, or be at least partially filled with a substance by incorporating an internal cavity or cavities, or being formed of a material with a substance in the matrix, or formed of the material having porosity or a material composition that will enable the link(s) to store a pharmaceutical agent or other substance necessary for patient treatment, and allowing such substance to pass from within the link, to outside the link. The substance thus can be on the chain, or in the chain, or part of the material of the chain, for delivery.

Substances can include solids, liquids or gases that can be released from the outside surfaces of the link(s), or from within the link(s), preferably in a controlled fashion after the implantation of the device. If the substance is monitoring equipment to provide one with monitored characteristics from within the body, such equipment can similarly be located within a link, or on the surface of a link, or make up a portion of the material of the link. For example, the monitoring substance could be a film capable of monitoring pre-selected characteristics, including for example temperature, stress, strain, and others.

The chain 10 preferably is at least partially covered with a shielding layer 60 as shown in FIG. 4 being a flexible, biocompatible polymer layer. Biocompatible surfaces lead to the success of a continuously-increasing number of polymer applications in the biomedical field. Surface chemistry controls numerous chemical and physiological properties of a polymer, including thromboresistance, biostability, lubricity, permeability, and abrasion resistance. Surface-modified polymers need to be well characterized in order to correlate the surface chemistry to the biofunctionality of the application.

The shielding layer 60 can comprise various polymers that take into account the design characteristics previously mentioned, as well as crystallization and calcification under cyclic loading. The polymer should not fracture or increase porosity within the mechanical environment of the chain 10. Silicon based rubbers have been used in these types of applications.

The surface of the chain 10 and/or of the shielding layer 60 can be clad partially or totally with a thin layer of hemocompatible carbon, for example turbostratic carbon. This cladding contributes to an improved hemocompatibility of the chain 10 and to a controlled tissue growth of the receiving organism.

The suturing layer 80 as shown in FIG. 4 provides a suitable material for suturing or otherwise attaching the chain 10 to the annulus tissue and promoting tissue growth therein. The suturing layer 80 can comprise a polyester knit or other fabric that is appropriate for suturing. The suturing layer 80 can comprise a biologically-compatible material such as, without limitation, Dacron (polyethylene terepthalate), polyester knit, PTFE knit, and ePTFE knit. The knit is beneficial because it provides a suitable surface for suture penetration as well as for tissue growth after implantation, reducing the risk of dehiscence.

The suturing layer 80 can also be treated with a biologically-compatible tissue growth factor or other medicament to aid in treating the attachment area. The present invention can reduce or eliminate the occurrence of systolic anterior motion (SAM), wherein the anterior leaflet of the mitral valve bulges into the left ventricular outflow track (LVOT) thereby obstructing blood flow into the aorta. Suture pull-out testing must be within the ranges required by international biomedical standards for this type of implants.

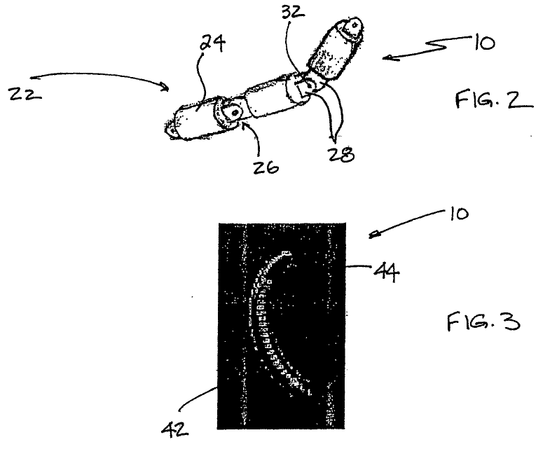

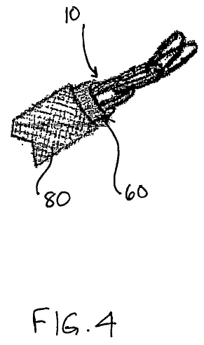

Attachment system 90 as shown in FIG. 5 can facilitate attachment of the chain 10 to annulus tissue. A number of attachment devices 92 can be positioned around the chain 10. The attachment device 92 can comprise various tissue-engaging devices, including, for example, needles, barbs, or hooks. Attachment devices 92 preferably incorporate a biologically compatible material such as, without limitation, stainless steel, titanium, or Nickel-Titanium alloy (Nitinol).

A chain holder dictates the initial shape of the chain, and the size of the implant. The surgeon should be able to suture the chain completely around the valve before retrieving the holder.

An embodiment of the present multilink chain 10 was tested in a physiological left heart simulator using human hearts. The results of the study showed that the ranges of geometrical variation used in this chain 10 induces geometries that do no present significant mitral regurgitastion in normal valves under physiological conditions.

The multilink chain 10 tested was a constant annular three-dimensional perimeter. To maintain this perimeter, the annulus in the model was constructed with a metallic multilink chain that allowed for a maximum change in linear length of 3%. Measuring a segment of the same length as that used in the model, in a maximum contractile and then distended state assessed this percentage.

The chain was then joined at the ends to form a circle that in its flat state had an approximate area of 7 cm². In the human heart, the annulus is not a perfect circle, and in the mid anterior leaflet area, the annulus tend to flatten out, generating a D-shaped configuration. To simulate this condition with the embedment of the present invention, a segment of the length where the anterior leaflet would be sutured was covered with resin to maintain a straight section in the perimeter (approximately 1.7cm). This D-shaped chain was then sutured onto a flexible elastic membrane. The membrane stretched and adhered to the modified atrial model.

The modification to the model included the addition of two metal rods that could be pushed forward and fixed in position. The ends of the rods were joined to the metallic annulus at the points corresponding to the center of the commissural areas. Then by pushing the rods forward, the annulus would deform. The contact points of the rods and the annulus protruded forward into the ventricle generating a saddle. The nature of the metallic chain ensured a soft curvature in the saddle shape.

The test was to simulate just a change in shape without displacement of the general structure. Thus, considering the eccentrically deformation when generating a saddle from a flat fixed perimeter ring, the mid point of the anterior side was fixed rigidly to the model, and the mid point of the posterior annulus was restrained by using a sliding bar mechanism that allowed for the structure to deform radially, but with no movement in the atrium-ventricular direction when force was applied on the rods. The metallic chain was covered with Dacron to facilitate the suturing of a natural valve on to it.

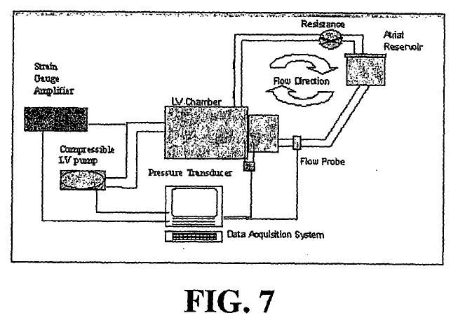

The model was capable of simulating a peak height of 1 cm, from the lowest part of the saddle to the peaks of the commissural areas. This implied an approximate height-to-diameter ratio of 1/3, which is the approximate relation found in a healthy human heart. Intermediate positions with lower height-to-diameter ratios can also be obtained with the model to simulate pathologic conditions in the heart. As observed in FIG. 7, this design assured soft curves in the three-dimensional saddle. The constant perimeter also implied a change in the two-dimensional projected area. The change is approximately of 21% when the maximum saddle curvature is applied. The change in projected area occurred naturally with the distortion of the three-dimensional shape.

Applicants reviewed the effects of a saddle-shaped annulus on mitral valve function and chordal force distribution in an in vitro study. Prior to Applicants' in vitro study, studies had concluded that the shape of the human mitral valve annulus is a three-dimensional saddle. The objective of Applicants' study was to investigate the effects of a saddle-shaped annulus on chordal force distribution and mitral valve function.

In a synopsis of the study, eleven human mitral valves were studied in a physiological left heart simulator with a variable shaped annulus (flat vs. saddle). Cardiac output and transmitral pressure were analyzed to determine mitral regurgitation volume. In six experiments, force transducers were placed on six chordae tendineae to measure chordal force distribution. Valves were tested in normal and pathophysiologic papillary muscle positions.

When comparing the flat and saddle-shaped configurations, there was no significant difference in mitral regurgitation volume 11.2±24.7% (p=0.17). In the saddle-shaped configuration, the tension on the anterior strut chord was reduced 18.5±16.1% (p<0.02), the tension on the posterior intermediate chord increased 22.3±17.1% (p<0.03), and the tension of the commissural chord increased 59.0±32.2% (p<0.01). Annular shape also altered the tensions on the remaining chords.

The study shows that annular shape alone does not significantly affect mitral regurgitation caused by papillary muscle displacement. A saddle-shaped annulus redistributes the forces on the chords by altering coaptation geometry, leading to an optimally balanced anatomic/physiologic configuration.

The following detail of the Applicants' study uses the following Key Terms, and Abbreviations:

| Key Term | Abbreviation | |
|---|---|---|
| Mitral Regurgitation | MA | Mitral Annulus |
| Chordal Force | PM | Papillary Muscles |
| Annulus Shape | MV | Mitral Valve |
| | IRB | Institutional Review Board |
| | FMR | Functional Mitral Regurgitation |
| | STDEV | Standard Deviation |
| | CTT | Chordae Tendineae Tension |
| | VASAC | Variable Annular Shape Atrial |
| | | Chamber |

The mitral annulus is a dynamic component of the mitral valve (MV) complex. Although the geometry and motion of the mitral annulus (MA) have been studied for several decades, there is still controversy over the exact geometry and dynamic characteristics of the MA including the origin of its shape. Sonomicrometry, magnetic resonance imaging, angiography, and two and three-dimensional echocardiographic techniques have been used to analyze the shape and dynamics of the MA in animal models and humans. Although there is still some disparity between measurements, current views tend to describe the annulus as a non-planar structure, which varies geometrically during the cardiac cycle.

The shape of the MA is described as a three-dimensional saddle because it resembles a non-planar, three-dimensional ellipse. In addition to its position, the area, the eccentricity, and the non-planarity or curvature of the MA vary during the cardiac cycle describing a dynamic structure.

Mitral annular geometry and dynamics have been studied in vivo in animals and humans, both in normal and pathologic subjects. Mitral annular geometry is an important factor in the diagnosis of MV prolapse. Changes in annular geometry and dynamics (2D-area, 2D-perimeter, saddle curvature, annular displacement, etc.) have been observed in patients with functional mitral regurgitation, FMR, acute ischemic mitral regurgitation, and different types of cardiomyopathies.

Although the exact origin and function of the shape of the MA is still unknown, studies have proposed that this shape may be important as part of the valve closure mechanism and in the distribution of stress on the mitral valve's anterior leaflet. The understanding of these mechanics may prove vital in the design of new surgeries involving chordal cutting and in the treatment of MV related pathologies. Annular shape is also considered in the design of different types of cardiac implants such as annuloplasty rings.

An objective of the Applicants' in vitro study was to compare mitral regurgitant volume and chordal force distribution in human MVs under saddle and flat annulus configurations in order to elucidate the importance of the saddle shape in MV mechanics. The experimental setup and procedure were not designed to imitate the complete function of the heart, but to isolate the effect of annular shape, while controlling other variables such as PM position, trans-mitral pressure, and flow rate.

Materials and Method

Mitral Valves

Four fresh human MVs from Emory University in Atlanta, Ga. and seven MVs from frozen hearts provided by Corazon Technologies in California were used in this study. The hearts from Emory University were obtained from heart transplant recipients with IRB approval following the guidelines for the protection of study volunteers in research. Hearts containing mitral valve pathology were excluded from the study. Valves with normal anatomical features and similar orifice areas (6.8±0.4cm²) were extracted.

The valves were extracted from the hearts preserving the complete mitral apparatus. During extraction, all chords that inserted into the leaflets or the annulus from the papillary muscles were preserved. The PMs were then wrapped with Dacron cloth maintaining the chordae distribution. The Dacron cloth was then sutured onto holding disks designed to attach to the Georgia Tech (Atlanta, Ga.) left heart simulator.

Anatomical Measurements

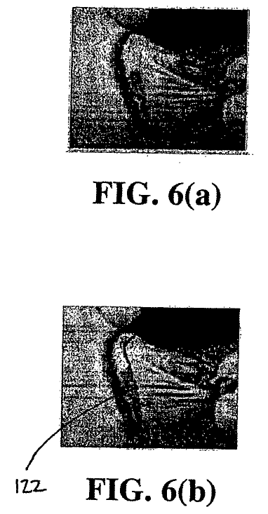

After extraction, six valves were selected for anatomical measurements. Only the last six valves were measured since this section of the procedure was not included in the initial protocol. The selected valves were sutured to a flexible membrane held by a rigid circular metallic ring. The membrane was used to hold the valve, while enabling the annulus to deform according to chordal lengths. The papillary muscles were positioned so that there was no slack on the chords inserting near the annulus of the valve. The lengths of the individual basal chords were measured from the origin in each papillary muscle to their insertion. Only chords inserting into the base of the leaflets were measured in order to analyze the geometry generated on the annulus when these chordae were under tension. The length of the chords was recorded in an insertion map of the valve. After these initial measurements, the flexible membrane was moved away from the papillary muscles to observe the geometry generated on the annulus as illustrated in FIG. 6(a). The membrane was then removed from the valve before the in vitro experiments. FIG. 6(b) shows the saddle configuration is present when the basal chords are extended as observed by the tracing 122 over the annulus.

In Vitro Flow Loop

The in vitro experiments were carried out in the modified Georgia Tech Left Heart simulator as shown in FIG. 7. This system is capable of physiologic and pathophysiologic flow and pressure waveforms. This simulator has been described in detail in previous studies.

Variable Shape Mitral Annulus Chamber (Flat-Saddle)

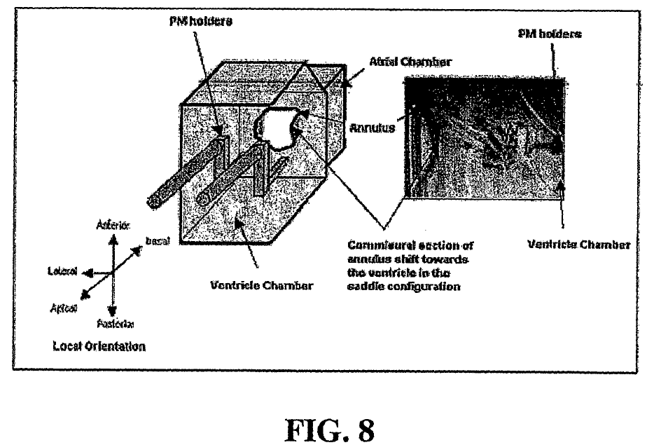

A variable annular shape atrial chamber (VASAC) was constructed to obtain the different annular geometries during the in vitro experiments. This chamber was used along with the Georgia Tech Left Heart simulator. The atrial chamber was constructed of transparent acrylic to enable visualization and echocardiographic imaging of the valve through a frontal window 5 cm away from the annulus. The annulus chain was composed of a multi-link chain which deformed three-dimensionally, but retained an approximately constant three-dimensional perimeter (maximum perimeter variation=3%). A 2 cm section of chain links were welded together to generate the D-shaped geometry characteristic of the mitral annulus orifice. Two straight control rods, connected at one end to the center of the commissural sections of the annulus, were used to modulate annular shape. Moving the control rods in the forward direction pushed these sections of the annulus forward, transforming the initially flat chain into a geometry similar to that of a saddle. The annulus was held fixed at the middle of its anterior section and was connected to a small metallic piston at the midpoint of the posterior section. Because of this design, when the rods were pushed forward to generate the saddle, the commissural section protruded into the ventricular cavity and the anterior section of the annulus was fixed in place as shown in FIG. 8.

Since the perimeter was constant, the posterior section of the annulus moved upward, reducing the septal-lateral diameter of the valve. The piston was used so that the posterior section of the annulus did not move apically, only septal-laterally. This variation in annular septal-lateral diameter is observed in the native mitral valve when going from a semi-flat structure in diastole to a three-dimensional saddle in systole. The whole chain is wrapped in a Dacron cloth allowing for extra support and the suturing of the valve. Annular geometry varied from a completely flat chain with an approximate orifice area of 6.8 cm² to a saddle-shaped geometry with saddle height of 9 mm. This resulted in a reduction of septal-lateral diameter of 3 mm, and a projected two-dimensional orifice area of 5.4 cm². A saddle height of 9 mm was selected because it represents an intermediate point within a disparity of measurements recorded in previous studies by other researchers. The annular area and annular area variation were within ranges observed clinically during the cardiac cycle.

Strain Gauge Transducers & Force Rods

Miniature C-shaped force transducers were used to measure the tension on individual chordae tendinea during the dynamic testing of the valve. The sensitivity (˜0.5 Newtons/Volt) and linearity of individual transducers was tested prior to and after each experiment. The minimal measurable difference in tension for these transducers was (0.5N/V*1.22 mv=6.1×10⁻⁴N) when coupled to the DAQ 1200 PCMCIA data acquisition card (National Instruments, TX, USA). The voltage baseline was zeroed immediately before testing.

The modified Georgia Tech left heart simulator used force rods, which attached to the sutured PMs, enabling the system to measure the total force applied on each PM. The rods were used to define the normal PM position of the valve for both the saddle and flat annulus configurations. This system was used as a reference, ensuring a comparable force on both PMs and maintaining approximately the same force conditions when changing annular shape. The construction and function of these rods has been described in a previous article.

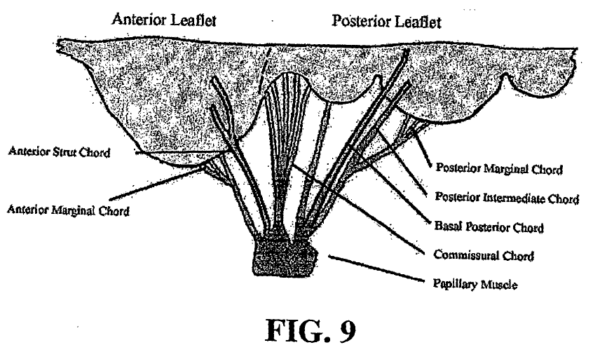

From the initial eleven valves, six were instrumented with forces transducers to measure chordae tendineae force distribution. Only six valves were instrumented because of C-ring availability. Six C-rings were individually sutured onto the following chords: anterior strut, anterior marginal, posterior intermediate, posterior marginal, basal posterior, and commissural (FIG. 9). It was not possible to attach all C-rings onto chords extending from a single PM because of spatial constraints. Chords were selected according to thickness and implantation feasibility. 5-0 sutures (braided silk, Ethicon, N.J., USA) were used to fasten the C-rings to the chords preventing the ring from slippage or detachment.

Echocardiographic Imaging

A Diagnostic Ultrasound System SSA-270A with a 3.75 MHz phased array transducer (Toshiba Corporation, Japan) was used to evaluate valve performance. Color Doppler velocity mapping was used to monitor valve function and regurgitation. The imaging depth of the transducer was 5 cm from the valve's annulus and reached an additional 6-8 cm downstream of the valve. Lateral views of the valve within the simulator were recorded in video. The videos and echo images of the valve can be observed on our website: http://www.bme.gatech.edu/groups/cfmg/web2/videos.html

Experimental Protocol

The atrial chamber containing the sutured MV was positioned in the left heart simulator. The PMs were attached to the force rods and the left heart simulator was then filled with 0.9% saline solution. All transducers and c-rings were zeroed and connected to an in-house interface box; which was then connected to a laptop computer. An in-house data collection program based on LabVIEW 5.0 software was used to store the flow, pressure and chordal force curves. This software stored the curves representing ten cardiac cycles for each variable. These were then averaged offline.

After preparing the system, the valve was placed in the defined normal PM position. The normal position was defined by:
Lateral Location: The papillary muscles arranged parallel to each other and directly aligned with the valve's annulus on each commissure. The commissural chords inserting in the annulus were vertically perpendicular to the annular plane.
Septal-lateral location: The rods were moved septal-laterally until an even extension of the commissural chords inserting into the annulus was observed. Normally, this point was a couple of millimeters below the annular height midpoint.
Basal-Apical location: The PM rods were moved towards the annulus to a point where slack was observed in all the chordae tendineae. The papillary force rods were zeroed at this location. Each force rod was pulled apically until a change in voltage of 0.02 volts (0.092 Newtons) was achieved for that particular rod. This was the minimal significant change that may be observed by the system. This defined a position with no slack or apparent tension on the chordae tendineae.

Valve function at this location was confirmed under pulsatile flow by observing appropriate leaflet coaptation.

The simulator ran under physiologic conditions with the valve in the normal position (Cardiac output: 5 l/min, Peak trans-mitral pressure: 120 mmHg, Cardiac rate: 70 BPM, Systolic duration: approx. 300 ms). Flow, tension, and pressure curves were saved for offline processing.

After the initial set of recordings with the flat annulus, the shape of the annulus was shifted to the saddle-shaped configuration. The PMs were then displaced apically to compensate for movement of the commissural section of the annulus into the ventricle. The force rods were used to ensure that the same force was applied on the PMs in both the flat and saddle configurations. All the previously described data acquisition and Doppler recordings were performed for this new annulus configuration using the same physiologic flow conditions. Both PMs were then moved 5 mm apically, 5 mm laterally, and 5 mm posteriorly from the normal position. This constitutes the symmetrically tethered PM position, which was used to induce mitral regurgitation. All the previously described data acquisition and Doppler recordings were performed at this new PM position using the same physiological flow conditions for both the flat and saddle annulus configurations.

Statistical Analysis

All data are reported as mean±1 standard deviation (STDEV) unless otherwise stated. Chordae tendineae forces were normalized for statistical comparisons using the flat annulus as control. Means were compared using two-tailed t tests for paired comparisons. Statistical analysis was carried out using Minitab (version 13.32) software. A P-value<0.05 was considered statistically significant.

Results

Anatomical Observations

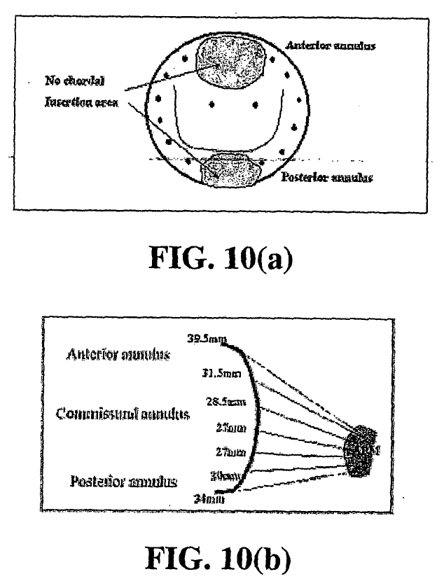

The anatomy of all valves showed dense chordae insertion in the commissural sections near the annulus when compared to the other areas of the MV. The midsections of the base of the anterior and posterior leaflets showed no direct insertions. The base of the anterior leaflet presented a larger area free from basal insertions when compared to the base of the posterior leaflet, as shown in FIG. 10(a) The chordae inserting into the central commissural areas adjacent to the annulus were significantly shorter than those inserting above and below this location (35.8% Anterior PM, 44.7% Posterior PM). These data are represented in FIG. 10(b). The MVs mounted onto the flexible membrane showed a saddle shape annular configuration when the PMs were moved away from the annulus (see FIG. 6(b)). The different lengths of the basal mitral chords and their insertion pattern are responsible for this saddle curvature.

In Vitro Experiments

Hemodynamics

All eleven valves were tested at 120±2 mmHg peak transmitral pressure and average flow rate of 5.03±0.1 l/min using the VASAC within the Georgia Tech left heart simulator.

In the defined normal PM position for both the flat and saddle annular configuration, the valves coapted well showing no regurgitant orifices along the coaptation line or leakage in the Doppler images. Apical posterior lateral displacement of the PMs induced tented leaflet geometries, reproducing configurations observed clinically. Mitral regurgitation was calculated by integrating the systolic negative volume in the flow curve, which included both closing and leakage volumes. Apical posterior lateral PM displacement was used to reproduce a severe pathological position. During apical posterior lateral PM displacement, the mean regurgitant volume was 9.8±3.84 ml for the saddle configuration and 10.9±3.52ml for the flat configuration. No significant difference in mitral regurgitation between the saddle and flat annular configurations was observed (p=0.165, n=11).

Chordae Tendineae Tension

Chordal tension was compared using peak systolic values for individual chords. Of the six valves tested with c-rings, data from the posterior marginal chord from valve 1 was discarded because of a strain gauge malfunction detected during the experiment and confirmed after the experiment with c-ring calibration. Peak systolic tension measurements under 0.1N were discarded, as they could not be distinguished from electrical crosstalk.

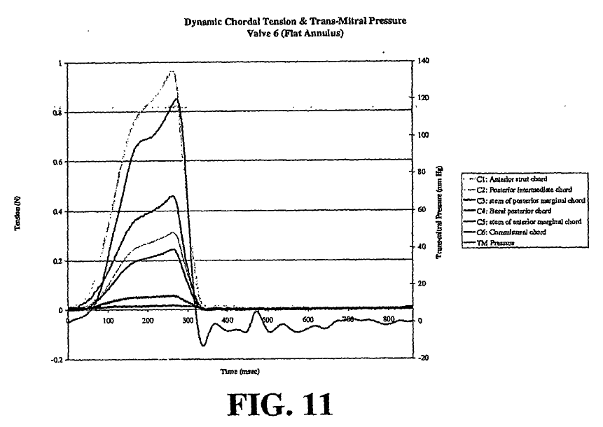

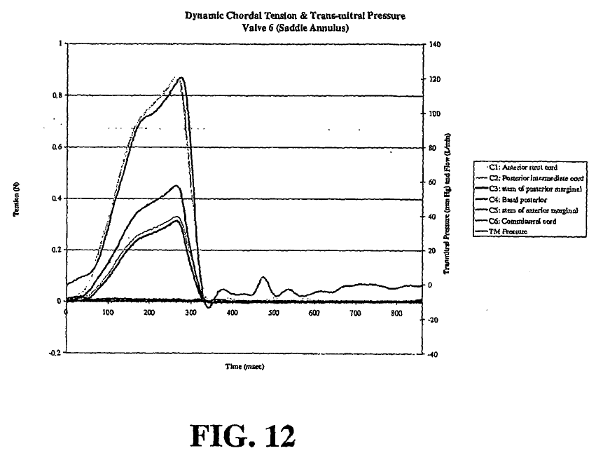

Chordae tenineae tension (CTT) curves were plotted against time during one cardiac cycle. Diastolic tension was considered as baseline for the dynamic CTT curves. As shown in FIGS. 11 and 12, CTT curves paralleled the tracing of the transmitral pressure curve. FIG. 11 are pressure and chordae tendineae tension curves for valve #6 in flat annulus configuration. FIG. 12 are pressure and chordae tendineae tension curves for valve #6 in saddled annulus configuration.

When comparing the systolic peak tensions on the different chords, the secondary chords (anterior strut and posterior intermediate chords) bore the larger loads on each of their respective leaflets when compared to the primary chords (anterior marginal and posterior marginal chords). The anterior strut chord had a tension 0.74±0.46 N higher than the anterior marginal chord, implying on average double the load observed on this marginal chord. The load on the posterior intermediate chord was 0.18±0.16N higher that the load on the posterior marginal chord. The commissural chord had a tension considerably smaller than that of the secondary chords, but close to that associated with the posterior basal chord.

Differences when comparing the peak systolic tensions in the two different annular configurations in the normal PM position, were measured as a percentage change using the flat annulus as a control. This eliminates to a certain extent the effects of the natural variation between valves. For all valves, the tension on the anterior strut chord was lower in the saddle configuration when compared to the flat configuration. The average difference of the force on this chord was 18.5±16.1%, being statistically significant (p<0.02, n=6). The average difference in the posterior intermediate chord was 22.3±17.1%, with higher tensions being present in all the valves for the saddle configuration. This result was statistically significant, with all valves showing the same trend in force variation (p<0.03, n=5). Although all valves showed an increase in tension for the posterior marginal chord in the saddle configuration, this change was not statistically significant (=O.12, n=4). Measurements on basal chords also showed an increase in tension in the saddle configuration for five valves. The average increase was 48.5±89.9%, although not statistically significant (p=O.12, n=6). In contrast, measurements on the commissural chord showed a decrease in tension in the saddle configuration for all valves. The average variation in force for this chord was 59.0±32.2% (p<0.01, n=5). For the anterior marginal chord, two valves showed a decrease in tension for the saddle configuration while four valves presented an increase in tension for this same configuration. The average increase in tension for the saddle configuration was 58.5±111.4%. However, because of the different tendencies in the results, this increase was not statistically significant (p=0.15, n=6).

When comparing the force distribution among the chordae tendineae, the flat annulus configuration showed a higher variability of tension between the different chords STDEV=±0.47N, when compared to the saddle configuration STDEV=±0.36N.

A summary of the peak systolic tensions on the chords and their variation from one annular configuration to the other are presented in Table 1.

TABLE 1

| | | PEAK SYSTOLIC TENSION | PEAK SYSTOLIC TENSION | | STATISTICAL |
|---|---|---|---|---|---|
| | NUMBER OF | FLAT ANNULUS | SADDLED ANNULUS | DIFFERENCE | SIGNIFICANCE |
| CHORD | SPECIMENS | (Newtons) | (Newtons) | (%) | P-Value |
| Anterior Strut | 6 | 1.22 ± 0.52 | 0.95 ± 0.35 | 18.5 ± 16.1 | 0.018 |
| Posterior Intermediate | 5 | 0.25 ± 0.14 | 0.30 ± 0.18 | −22.3 ± 17.1 | 0.022 |
| Posterior Marginal | 4 | 0.03 ± 0.04 | 0.06 ± 0.05 | −137.8 ± 188.6 | 0.12 |
| basal posterior | 6 | 0.19 ± 0.10 | 0.31 ± 0.25 | 48.5 ± 89.9 | 0.122 |
| Anterior Marginal | 6 | 0.31 ± 0.17 | 0.35 ± 0.16 | −58.5 ± 111.4 | 0.145 |
| Commissural | 5 | 0.17 ± 0.18 | 0.11 ± 0.20 | 59.0 ± 32.3 | 0.008 |

Discussion

Mitral Annulus Shape

The results describe increases in length of the basal chords from the commissural to the anterior and posterior segments of the annulus, which are larger than those determined by Pythagorean relations. When the chordae tendineae are extended and the annulus is relatively free to deform, the MA generates a saddle-shaped configuration. Chordae tendineae lengths are approximately constant during the cardiac cycle, and there is a higher density of basal chordae inserting into the commissural section of the annulus. As a consequence, when under systolic pressure the mitral valve is pushed backwards. The free posterior and anterior sections of the annulus deflect into the atrium, while the commissural sections are relatively held in place by the PMs and corresponding chords. Annular flexing/bending and anatomical relations may partially explain the saddle shape of the annulus, but the shape of the mitral annulus is not a simple symmetric elliptical saddle, but a complex asymmetrical saddle structure. Other phenomenon such as myocardial contraction, aortic expansion, PM contraction and ventricular motion may affect the shape of the annulus. Therefore, the complex inter-relation between these mechanisms warrants further investigation.

Valve Function

Geometrical variations in mitral annular shape have been observed in patients with pathologies such as functional mitral regurgitation, hypertrophic obstructive and dilated cardiomyopathy, and ischemic mitral regurgitation. Loss of saddle curvature has been described as a possible cause for mitral regurgitation in animal and human studies. Patients with FMR showed loss of curvature in the saddled annulus, which subsequently may increase annular area because of reduced flexing. In-vitro studies have shown that only increases in projected area over a factor of 1.75 will induce mitral regurgitation without PM displacement. Therefore, area changes associated with a loss of curvature are not sufficient to induce regurgitation. The loss in curvature in FMR patients may be related to changes in ventricular and PM dynamics since loss of annular displacement, curvature, and dynamical change have also been observed in regurgitation associated pathologies. Therefore, loss of annular curvature and regurgitation may not hold a cause consequence relationship, but both may have similar origins. This may explain why variation of annular shape alone (flat-saddle) did not induce mitral regurgitation as represented by the results of this study.

Chordae Tendineae Force Distribution

The results showed for both configurations, force distributions characterized by the secondary chords carrying most of the load on their respective leaflet. This phenomenon has been observed and analyzed by other researchers. The saddle configuration showed a more evenly distributed force as illustrated by the variance of the tensions on the different chords. This phenomenon may also be observed in FIGS. 11 and 12 where the tension curves for the different chords are closer for the saddle configuration. Therefore, the saddle configuration optimizes the force distribution on the valve since a larger number of chords are extended and the load is divided more evenly among them.

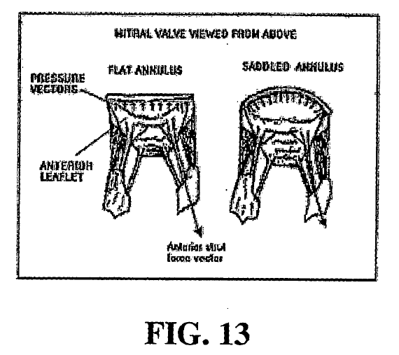

The forces on the MV and its apparatus are determined by several factors: valve geometry, leaflet area, transmitral pressure, and contact forces along the coaptation line. In parallel, leaflet curvature has been shown to be important in valve mechanics, as billowing (primary curvature) and saddle curvature (secondary curvature) may reduce stress on leaflets. The reduction in force for the anterior strut chord may be explained by a redistribution of the force vectors caused by pressure because of the secondary curvature generated by the saddle (FIG. 13). Since pressure acts perpendicularly to the surface, due to the secondary curvature of the saddle, more force vectors are directed towards the commissural direction and less in the apical direction. The anterior strut chord is directed mostly in the apical direction implying that its tension will be reduced if the apical force component generated by pressure decreases. Chords extending in other directions must then balance the new redirected components of the force. These redirected vectors may explain the increase in force on the other chords.

The peak systolic tension of the anterior marginal chord did not vary significantly, and this variation followed different trends for different valves. The peak systolic tension values for the posterior marginal chord were small and near the limit of the c-ring crosstalk range, all the values above the crosstalk threshold followed the same trend of increased force. This variability may be explained by the fact that marginal chords insert on the outer edge of the leaflet implying that tension on this chord is predominantly determined by transmitral pressure, contact forces, and coaptation line geometry and location. Leaflet curvature may have a lesser effect on marginal chords than on secondary chords.

During coaptation, the posterior leaflet central scallop is extended for the most part and it is smaller than the anterior leaflet; therefore, the effects of force redistribution are probably less than those seen on the anterior leaflet. Meanwhile, the relative distance from the PMs to the anterior and posterior segments of the annulus is increased by the saddle geometry. This increase in length, coupled with the decreased effect of the saddle curvature may explain the increased tension on the posterior intermediate chord. The basal posterior chord inserts directly above the posterior section of the annulus. The increased distance between the PMs and the annulus may account for the increased tension on this particular chord in the saddle configuration.

The clinical relevance of this study lies both in the cardiac implant field as well as in the surgical field. Considering the results, which clearly show that the shape of the annulus alters the force distribution among the chords, implants such as annuloplasty rings must consider the effect of their implantation on chordal force distribution. Increased tension on the chords under cyclic loading implies under engineering standards a lower life expectancy for the chords because of possible tissue damage. On the other hand, reduction in force may increase the life expectancy of the chord, but if severe, this reduction may induce negative effects on valve function. Several authors have proposed chordal cutting as an alternative procedure for pathologies such as ischemic mitral regurgitation. For the most part surgeons have observed that cutting the primary (marginal chords) induces severe regurgitation, but that in some pathologies cutting the secondary (intermediate chords) may decrease leaflet tenting leading to better coaptation and decreasing regurgitation. Some surgeons are reluctant to use these procedures because cutting the large secondary chords may induce significantly higher loads on other chords that may eventually fail due to structural deterioration. As shown in our results, the secondary chords do carry the highest loads and therefore are structurally relevant to mitral valve function. Therefore, the increased load generated by cutting these chords warrants further detailed/fundamental studies, both in vitro and in vivo.

Limitations

There are several limitations associated with both the apparatus and the procedure. An initial limitation of this study was the limited population of human MVs. Unfortunately, this is the situation involving any study that utilizes human organs. Even though the chordae were carefully selected under a strict characterization protocol, their size and ramification varied from valve to valve. MV leaflet size and coaptation geometry also varied from valve to valve. Coaptation line location and geometry also varied from valve to valve although a standard normal position was used. Because of this natural variability and the reduced population, the standard deviations for the results were high.

The left ventricle heart simulator has several limitations, but it has been used successfully in several studies. Although the pressure and flow conditions generated in this loop are physiological, it does not reproduce phenomenon such as ventricular, atrial, or papillary muscle contractions since it is a rigid simulator. More important to this study, we used a static annulus, which did not vary in size or shape during the cardiac cycle. The VASAC was designed to mimic geometrical conditions found during peak systole when the saddles curvature is at its maximum. Annular motion, which has been to some extent related to mitral regurgitation was not modeled.

Measurement of tensions using the c-ring transducers had some technical limitations. The weight of these transducers even if minimal compared to other transducers may affect readings of absolute tension on the chords. However, for dynamic changes in tension, the variation generated by the weight of the transducers is not significant especially in chords with high loads. The level/noise/crosstalk range is high for these transducers when measuring the forces on the minor chords. Although the crosstalk margin persisted, acquiring data over ten cardiac cycles and averaging the readings over the cardiac cycle reduced level/noise error.

Future Work

Considering the limitations of the current VASAC, Applicants intend to construct an annulus model that will self modulate its annular shape during the cardiac cycle. Data acquired with the new model will be compared to present data to observe the difference in force distribution between a flexible annulus and a rigid annulus. This comparison could clarify the difference in force distribution generated on the mitral valve after the implant of a rigid annuloplasty ring. The protocol will be modified so that the chordae tendineae cross-sectional area is available, this will allow us to calculate the stress on the different chords and therefore obtain valuable information about chordae tendineae failure mechanics. Another important factor that should be simulated in future work is PM function including contractility. Therefore, a larger range of physiological and pathological conditions related to PM function could be studied. Finally, after having a broad understanding of normal MV mechanics, abnormal valves may be studied in order to understand the mechanics of their pathologies. Surgical procedures could be reproduced in vitro using these diseased valves, to observe the effectiveness of the proposed correction methods.

Conclusions

Although not all conditions of mitral annular mechanics were replicated, this study simulated the effect of changing annular geometry from a flat ring to a three-dimensional saddle on chordae tendineae force distribution and mitral regurgitation due to PM displacement. A saddle-shaped geometry reduces mitral annulus orifice area by decreasing the septal-lateral diameter of the valve. However, annular shape alone does not significantly affect mitral regurgitation due to papillary muscle displacement because of the MVs redundant leaflet design.

Annular geometry directly affects tension on the basal chords by varying the relative distance from their insertion point to the PMs. The tension on the anterior strut chord is significantly reduced by a saddle-shaped annular geometry because the secondary curvature of the anterior leaflet causes redirection of the force vectors generated by pressure. The natural configuration of the MA is that of a three-dimensional saddle. In this configuration more chords are extended and a secondary curvature in the leaflets is induced. Therefore, the saddle-shaped annulus redistributes the forces on the chordae tendineae leading to a more even distribution of tensions among the chords.

While the invention has been disclosed in its preferred forms, it will be apparent to those skilled in the art that many modifications, additions, and deletions can be made therein without departing from the spirit and scope of the invention and its equivalents as set forth in the following claims.

What is claimed is:

1-38. (canceled)

39. An annular prosthesis for a heart valve comprising a chain having a plurality of links, wherein upon implantation, the prosthesis can reinstate the proper shape and dimensions of the valve annulus, the prosthesis implanted without necessity of suture stabilizers or placation bands.

40. The annular prosthesis of claim 39, wherein upon implantation, the prosthesis generates a saddle-shaped geometry and deforms three-dimensionally, while retaining an approximately constant three-dimensional perimeter.

41. The annular prosthesis of claim 40, wherein upon implantation, the prosthesis has a saddle height to commissural diameter ratio in the range from approximately 0 to approximately 1/3.

42. The annular prosthesis of claim 39, wherein upon implantation, the prosthesis retains an approximately constant three-dimensional perimeter, with a maximum variation in perimeter of less than approximately 10%.

43. The annular prosthesis of claim 42, wherein the maximum variation in perimeter is less than approximately 3%.

44. The annular prosthesis of claim 39, wherein upon implantation, the prosthesis maintains a normal chordal force distribution as its bending is dominated by its mechanical environment.

45. An annuloplasty ring for a heart valve comprising a prosthesis, wherein upon implantation, the prosthesis maintains a normal chordal force distribution during the cardiac cycle as its bending is dominated by its mechanical environment.

46. An annuloplasty ring for a heart valve comprising a prosthesis, wherein upon implantation, generates a saddle-shape geometry, and deforms three-dimensionally, while retaining an approximately constant three-dimensional perimeter.

47. The annuloplasty ring of claim 46, wherein the prosthesis has a saddle height to commissural diameter ratio in the range from approximately 0 to approximately 33%.

48. The annuloplasty ring of claim 47, wherein the prosthesis has a saddle height to commissural diameter ratio of approximately 25%.

49. A supporting prosthesis for repairing pathological alterations of valves of the heart comprising:
a chain having a plurality of links;
shaping means, wherein upon implantation to annulus tissue, the chain generates a variable saddle-shaped geometry during the cardiac cycle, and deforms three-dimensionally, to reconstruct the shape of a valve, while maintaining the dynamics of the valve through appropriate flex and bend as to allow the valve to thereafter function correctly.

50. The supporting prosthesis of claim 49, wherein upon implantation, the chain maintains a normal chordal force distribution as its bending is dominated by its mechanical environment.

51. A method of repairing pathological alterations of a heart valve comprising the steps of:
providing a chain having a plurality of links;
implanting the chain in a minimally invasive procedure; and
reconstructing with the chain the shape of a valve, while maintaining the dynamics of the valve through appropriate flex and bend as to allow the valve to thereafter function correctly.

52. The method according to claim 51, comprising the further step of directly implanting the chain to annulus tissue, wherein the chain is directly implanted to the annulus tissue without necessity of suture stabilizers or placation bands.

53. The method according to claim 51, wherein the step of implanting comprises suturing the chain directly to the annulus tissue.

54. The method according to claim 51, wherein the step of implanting comprises clipping the chain directly to the annulus tissue.

55. The method according to claim 51, wherein the step of implanting the chain in a minimally invasive procedure occurs with a beating heart.

56. The method according to claim 51, comprising the further step of maintaining with the chain a normal chordal force distribution, as its bending is dominated by its mechanical environment.

57. The method according to claim 51, comprising the further steps of generating with the chain a saddle-shaped geometry; and
deforming the chain three-dimensionally, while retaining an approximately constant three-dimensional perimeter.

58. The method according to claim 57, wherein the chain has a saddle height to commissural diameter ratio in the range from approximately 0 to approximately 1/3.

59. The method according to claim 51, wherein the step of implanting the chain in a minimally invasive procedure comprises implanting the chain during a percutanueous approach.

60. The method according to claim 51, wherein the step of implanting the chain in a minimally invasive procedure comprises implanting the chain during a endovascular approach.
